(19) United States
(12) Patent Application Publication
(10) Pub. No.: US 2024/0277605 A1
(43) Pub. Date: Aug. 22, 2024

(54) COMPOSITION COMPRISING A PARTICULAR ANTIOXIDANT, A SPECIFIC FATTY ACID ESTER, A BIFIDOBACTERIUM SPECIES LYSATE AND A YEAST EXTRACT

(71) Applicant: L'Oreal, Paris (FR)

(72) Inventors: Laurence Richet, Saint Ouen (FR); Marie-Cécile Degoul, Saint Ouen (FR); Edine Ahbich, Saint Ouen (FR); Angélique Jacquet, Saint Ouen (FR); Aurélie Phelipot, Saint Ouen (FR)

(73) Assignee: L'Oreal, Paris (FR)

(21) Appl. No.: 18/292,771

(22) PCT Filed: Jul. 22, 2022
(86) PCT No.: PCT/EP2022/070672

§ 371 Date: Jan. 26, 2024

(30) Foreign Application Priority Data
Jul. 30, 2021 (FR) 2108293

(51) Int. Cl. A61K 8/99; A61K 8/49; A61K 8/60; A61K 8/67; A61K 8/9728; A61Q 5/00; A61Q 19/00

(52) U.S. Cl. CPC A61K 8/99; A61K 8/4993; A61K 8/60; A61K 8/676; A61K 8/9728; A61Q 5/006; A61Q 19/00

(57) ABSTRACT

The present invention relates to a composition comprising a combination of an antioxidant, of a specific fatty acid ester, a Bifidobacterium species lysate and a yeast extract. The invention also relates to a method for the cosmetic treatment of keratin materials, in particular human keratin materials, such as the scalp and the hair, employing said composition. The present invention also relates to the non-therapeutic cosmetic use of said composition for maintaining, indeed even restoring, the balance of the microbial flora of the scalp. The invention additionally relates to a device comprising one or more containers which comprises (comprise) the composition according to the invention.

The present invention relates to a composition comprising a combination of a particular antioxidant, of a specific fatty acid ester, a Bifidobacterium species lysate and a yeast extract.

The invention also relates to a method for the cosmetic treatment of keratin materials, in particular human keratin materials, such as the scalp and the hair, employing said composition.

The present invention also relates to the non-therapeutic cosmetic use of said composition for maintaining, indeed even restoring, the balance of the microbial flora of the scalp.

The invention additionally relates to a device comprising one or more containers which comprises (comprise) the composition according to the invention.

Human skin, and in particular the scalp, consists of two compartments, namely a deep compartment, the dermis, and a superficial compartment, the epidermis.

The skin has a major role in protection from external attacks, such as environmental attacks, attacks from the weather (heat, cold, UV, tobacco, and the like), pollution, allergens, pathogenic germs, mechanical attacks (depilation, shaving, abrasive scrubbing) and chemical attacks (detergents). This property, known as the barrier function, is primarily ensured by the uppermost layer of the epidermis, namely the horny layer known as the stratum corneum.

The skin also represents a complex ecosystem on which several types of microorganisms, such as bacteria and fungi, proliferate. These microorganisms constitute the cutaneous flora, also known as microbial flora of the skin. Within this ecosystem, a balance exists between the various species of microorganisms.

To date, more than 500 bacterial species have been detected on healthy skin, with more than 2 million genes. Each cm² of skin is populated by approximately 106 bacteria.

The following in particular are distinguished:
the commensal beneficial resident flora constituted of the microorganisms conventionally proliferating on healthy skin, continuously while drawing their nutrients from the skin, and bringing known benefits to the skin;
the transitory flora, present on the skin under abnormal conditions, for example by contact with soiled elements, and which may become pathogenic in the event of proliferation.

In cases where the barrier is detrimentally affected or when the balance between commensals and pathogenic agents is disturbed, for example subsequent to an external attack, a detrimental change in the quality of the skin and/or skin disorders may result therefrom, such as, for example, instances of stinging, tightness, itching, and the like.

It is apparent, moreover, that the quality of the skin barrier is affected daily as a result of external attacks of the type of irritant agents (surfactants, acids, bases, oxidizing agents, reducing agents, concentrated solvents), mechanical stresses (rubbing, shaving) or thermal or weather imbalances (cold, dryness).

Thus, the (re)colonization of the skin by beneficial microorganisms of the cutaneous commensal flora appears to be fundamental for the physiology and the immunity of the skin, in particular by virtue of their actions on limiting the adhesion of microorganisms, in particular pathogenic microorganisms, the restoration/recovery of the barrier function in particular following an external attack, such as mentioned above.

Cosmetic solutions are already known for acting on the cutaneous microbial flora, such as the use of compositions containing probiotics or the use of pullulan or of one of its derivatives or of its combination with a polysaccharide, to maintain and/or preserve the balance of the cutaneous and/or mucosal microbial flora.

However, these solutions are difficult to transpose to use on the scalp, in particular for the reasons mentioned below.

One of these reasons lies in the fact that the microbial flora of the scalp is different from that of the skin of the face or of the body; the cutaneous commensal flora (skin of the face or of the body) is mainly composed of 5 bacteria (Cutibacterium acnes/Corynebacterium/Staphylococcus epidermis/Streptococcus/Neisseria). The microorganisms present on the scalp are, for their part, mainly represented by 3 microorganisms: Malassezia restricta, Staphylococcus epidermis and Cutibacterium acnes (also known as Propionibacterium acnes), which represent the bulk of the microbial population of the scalp.

In addition, although two bacteria (Staphylococcus epidermis and Cutibacterium acnes) are common to the floras of the scalp and of the skin, their relative proportions are different thereon: these two bacteria represent 99% of the bacteria of the flora of the scalp, whereas they represent only 75% of the bacteria of the flora of the skin of the face.

The flora of the scalp thus exhibits a much lower diversity than that of the skin of the face.

Moreover, the presence of keratin fibres (hair) on the scalp in greater density and/or in greater length than on the skin of the face or of the body (non-head hair) induces an additional constraint. This is because the cosmetic treatments used must not have negative effects on the appearance and/or on the feel of the hair; in particular, they must not result in a greasy effect on the hair or the roots and/or in an unpleasant feel and/or an undesired appearance of the hair.

Another point lies in the fact that the properties expected for a composition for the scalp may be different from those provided by a composition for the skin; the scalp may exhibit instances of discomfort, such as stinging or feelings of heating, for example due to an excessively high presence of dandruff, which are consequences of the presence of an unusual amount of Malassezia restricta; this microorganism is present naturally on a healthy scalp but its excessive proliferation can cause annoyance. There thus exists a real need to make available a hair composition which makes it possible to maintain or to restore the balance of the very particular microbial commensal flora of the scalp, while making it possible to reduce, indeed even to eliminate, the undesirable effects of discomfort of the scalp, such as stinging, itching and/or feeling of heating, and while retaining, on the hair and the roots, a clean, non-greasy and non-tacky feel matching the expectations of consumers with regard to products of this type.

It has now been discovered that a composition comprising the combination of at least one antioxidant chosen from ascorbic acid and its derivatives, of at least one specific fatty acid ester, of at least one Bifidobacterium species lysate and of at least one yeast extract makes it possible to achieve the objectives set out above, and in particular to provide a composition capable of maintaining, indeed even restoring the balance of the microbial flora of the scalp, while providing a soothing effect to the scalp and while limiting the oxidation of the sebum.

A subject-matter of the present invention is thus a composition comprising:
(i) one or more antioxidants chosen from ascorbic acid and its derivatives,
(ii) one or more oxyalkylenated esters of fatty acids and of sorbitan,
(iii) one or more microorganisms of the genus Bifidobacterium species, one of its fractions, one of its metabolites or their mixtures, the microorganism(s) being in the lysate form, and
(iv) one or more extracts of yeast of the genus Saccharomyces.

The composition according to the invention makes it possible in particular to maintain, indeed even to restore, the balance of the commensal microbial flora of the scalp, while providing an anti-oxidizing effect capable of reducing the irritation and itching related to the oxidation of the sebum.

The composition according to the invention exhibits a soothing and anti-oxidizing effect on the scalp. It also limits the adhesion of the particles resulting from pollution to the scalp and/or to the hair.

By virtue of the choice of specific non-ionic surfactants present in the composition, it is possible to obtain a composition which is particularly stable over time and which will retain its properties over a long duration. Furthermore, the presence of these oxyalkylenated esters of fatty acids and of sorbitan confers, on the composition according to the invention, an enhanced soothing effect on the scalp.

The microbiome of the skin, and in particular of the scalp, thus strengthened, indeed even rebalanced, after application of the composition of the invention, is better able to defend itself against exterior attacks and to combat discomfort.

The composition according to the invention additionally makes it possible to obtain a beautiful head of hair, with hair exhibiting an appearance which is neither greasy nor dry, and with a clean and natural feel.

The composition according to the invention also makes it possible to impart conditioning properties to the hair, particularly in terms of improving the volume of the hair. In addition, the hair has a natural, clean feel and look.

The composition according to the invention is advantageously clear, indeed even transparent, which gives it an aesthetic appearance particularly attractive to users.

It can in addition be provided with a gel texture or else a texture of serum type, which facilities the application thereof on the scalp (product which does not flow, which can gather together).

The present invention additionally relates to a method for the cosmetic treatment of keratin materials, in particular human keratin materials, such as the scalp and the hair, comprising the application, to said keratin materials, of a composition as defined above.

In particular, the cosmetic treatment method according to the invention makes it possible to maintain and/or to restore the balance of the microbial flora of the scalp.

The present invention also relates to the non-therapeutic cosmetic use of a composition as defined above for maintaining, indeed even restoring, the balance of the microbial flora of the scalp.

The invention additionally relates to a device comprising one or more containers comprising the composition according to the invention.

Other subject-matters, characteristics, aspects and advantages of the invention will become even more clearly apparent on reading the description and the example which follows.

In that which will follow, and unless otherwise indicated, the limits of a range of values are included within this range, in particular in the expressions “of between” and “ranging from . . . to . . . ”.

Moreover, the expression “at least one” used in the present description is equivalent to the expression “one or more”.

Furthermore, the term “composed of X OE” is understood to mean, within the meaning of the present invention, an oxyethylenated compound comprising X oxyethylene units per molecule.

(i) The Antioxidants Chosen from Ascorbic Acid and its Derivatives

The composition according to the present invention comprises one or more antioxidants chosen from ascorbic acid and its derivatives.

The term “antioxidant” is understood to mean, within the meaning of the present invention, an agent which slows down or prevents the oxidation of other chemical substances on contact with it.

The antioxidant(s) are chosen from ascorbic acid and its derivatives, in particular its esters, especially ascorbyl palmitate, magnesium ascorbyl phosphate and ascorbyl glucoside, and their mixtures.

Preferably, the antioxidant is ascorbyl glucoside.

The total content of the antioxidant(s) (i) present in the composition according to the invention preferably ranges from 0.05% to 10% by weight, and more preferentially from 0.1% to 10% by weight, better still from 0.5% to 10% by weight, even better still from 0.7% to 5% by weight, indeed even from 0.8% to 3% by weight, with respect to the total weight of the composition.

In a preferred alternative form of the invention, the antioxidant (i) is ascorbyl glucoside and the total content of the ascorbyl glucoside present in the composition preferably ranges from 0.05% to 10% by weight, and more preferentially from 0.1% to 10% by weight, better still from 0.5% to 10% by weight, even better still from 0.7% to 5% by weight, indeed even from 0.8% to 3% by weight, with respect to the total weight of the composition.

### (ii) The Oxyalkylenated Esters of Fatty Acids and of Sorbitan

The composition according to the present invention additionally comprises one or more oxyalkylenated esters of fatty acids and of sorbitan.

The oxyalkylene units of the ester(s) of fatty acids and of sorbitan (ii) are more particularly oxyethylene or oxypropylene units or their combination. Preferably, the ester(s) of fatty acids and of sorbitan (ii) used in the present invention are oxyethylenated.

The number of moles of ethylene oxide and/or propylene oxide advantageously ranges from 1 to 250, preferably from 1 to 100, more preferentially from 1 to 50 and better still from 1 to 20.

Preferably, the oxyalkylenated ester(s) of fatty acids and of sorbitan (ii) are preferentially chosen from esters of saturated or unsaturated, linear or branched, C₈ to C₃₀ fatty acids and of sorbitan, comprising from 1 to 20 oxyethylene units, and their mixtures, and more preferentially from esters of preferably linear and saturated C₈ to C₁₈ fatty acid and of sorbitan, comprising from 4 to 20 oxyethylene units, and their mixtures, better still from esters of preferably linear and saturated C₁₀-C₁₄ fatty acid and of sorbitan, comprising from 4 to 10 oxyethylene units.

Such compounds are known in particular under the name of polysorbates. They are, inter alia, sold under the name Tween by Uniqema. Mention may, for example, be made of polyoxyethylene (40E) sorbitan monolaurate (polysorbate 21), sold under the name Tween 21, polyoxyethylene (200E) sorbitan monolaurate (polysorbate 20), sold under the name Tween 20, polyoxyethylene (200E) sorbitan monopalmitate (polysorbate 40), sold under the name Tween 40, polyoxyethylene (200E) sorbitan monostearate (polysorbate 60), sold under the name Tween 60, polyoxyethylene (40E) sorbitan monostearate (polysorbate 61), sold under the name Tween 61, polyoxyethylene (200E) sorbitan tristearate (polysorbate 65), sold under the name Tween 65, polyoxyethylene (200E) sorbitan monooleate (polysorbate 80), sold under the name Tween 80, polyoxyethylene (50E) sorbitan monooleate (polysorbate 81), sold under the name Tween 81, and polyoxyethylene (200E) sorbitan trioleate (polysorbate 85), sold under the name Tween 85.

Preferably, the fatty acid of the oxyalkylenated ester of fatty acid and of sorbitan is a saturated fatty acid.

The oxyalkylenated ester(s) of fatty acid and of sorbitan (ii) are preferably chosen from polyoxyethylene (40E) sorbitan monolaurate, polyoxyethylene (200E) sorbitan monolaurate, polyoxyethylene (200E) sorbitan monopalmitate, polyoxyethylene (200E) sorbitan monostearate, polyoxyethylene (40E) sorbitan monostearate, polyoxyethylene (200E) sorbitan tristearate, polyoxyethylene (200E) sorbitan monooleate, polyoxyethylene (50E) sorbitan monooleate and polyoxyethylene (200E) sorbitan trioleate; preferentially, they are chosen from polyoxyethylene (40E) sorbitan monolaurate, polyoxyethylene (200E) sorbitan monolaurate and their mixtures. More preferentially, the oxyalkylenated ester of fatty acid and of sorbitan (ii) is polyoxyethylene (40E) sorbitan monolaurate.

The total content of the oxyalkylenated ester(s) of fatty acids and of sorbitan (ii) which are present in the composition according to the invention preferably ranges from 0.05% to 10% by weight, more preferentially from 0.1% to 5% by weight and better still from 0.2% to 2% by weight, with respect to the total weight of the composition.

In a preferred alternative form of the invention, the oxyalkylenated ester of fatty acid and of sorbitan (ii) is polyoxyethylene (40E) sorbitan monolaurate and the total content of polyoxyethylene (40E) sorbitan monolaurate present in the composition preferably ranges from 0.05% to 10% by weight, more preferentially from 0.1% to 5% by weight and better still from 0.2% to 2% by weight, with respect to the total weight of the composition.

(iii) The Microorganisms of the Genus Bifidobacterium Species

The composition according to the present invention additionally comprises one or more microorganisms (iii) of the genus Bifidobacterium species, one of its fractions, one of its metabolites or their mixtures, the microorganism(s) being in the lysate form.

The term “metabolite” is understood to mean, within the meaning of the present invention, any substance resulting from the metabolism of the microorganism(s) (iii) of the genus Bifidobacterium species and also having an effectiveness in protecting the scalp from oxidative stress.

The term “fraction” is understood to mean, within the meaning of the present invention, a fragment of the microorganism(s) (iii) having an effectiveness in protecting the scalp from oxidative stress, by analogy with the whole microorganism(s) (iii).

The microorganism(s) (iii) of the genus Bifidobacterium species are present in the composition of the invention in the lysate form.

A lysate commonly denotes a material obtained after the destruction or the dissolution of the biological cells of the microorganism(s) (iii) by a phenomenon referred to as cell lysis, thus causing the release of the intracellular biological constituents naturally contained in said cells of the microorganism (iii).

In the context of the present invention, the term “lysate” is used without preference to denote all of the lysate obtained by lysis of the microorganism (iii) or only a fraction of the lysate. The lysate used is thus formed completely or partially of the intracellular biological constituents and of the constituents of the walls and cell membranes.

More specifically, the lysate contains the cellular cytoplasmic fraction containing enzymes, such as lactic acid dehydrogenase, phosphatases, phosphoketolases or transaldolases, and the metabolites. By way of example, the constituents of the cell walls are in particular peptidoglycan, murein or mucopeptide and teichoic acid, and the constituents of the cell membranes are composed of glycerophospholipids.

This cell lysis can be carried out by various techniques, such as, for example, osmotic shock, thermal shock, ultrasound, or under a mechanical stress, such as centrifugation. More particularly, the lysate of the microorganism(s) (iii) can be obtained according to the technology described in U.S. Pat. No. 4,464,362 and in particular according to the following protocol.

The microorganism(s) (iii) of the genus Bifidobacterium species are preferably cultured under anaerobic conditions in an appropriate culture medium, for example according to the conditions described in the documents U.S. Pat. No. 4,464,362 and EP 0 043 128. When the stationary phase of development is reached, the culture medium can be inactivated by pasteurization, at a temperature ranging, for example, from 60 to 65° C., for 30 minutes. The microorganism(s) thus formed are subsequently collected by a conventional separation technique, such as membrane filtration or centrifugation, and are then resuspended in a sterile physiological solution of NaCl.

The lysate of these microorganisms can subsequently be obtained by ultrasonic disintegration of the solution obtained above, so as to release the cytoplasmic fractions, the cell wall fragments and the products resulting from the metabolism. All the components, in their natural distribution, are subsequently stabilized in a weakly acidic aqueous solution. The total content of active material(s), present in the lysate obtained by this process, preferably ranges from 0.1% to 50% by weight, more preferentially from 1% to 20% by weight, and better still this content is 5% by weight, with respect to the total weight of the lysate.

The lysate can be used in various forms, in particular in the form of a solution or in pulverulent form.

Preferably, the microorganism(s) (iii) of the genus Bifidobacterium species are chosen from the following species: Bifidobacterium longum, Bifidobacterium bifidum, Bifidobacterium breve, Bifidobacterium animalis, Bifidobacterium lactis, Bifidobacterium infantis, Bifidobacterium adolescentis, Bifidobacterium pseudobacterium and their mixtures.

More preferentially, the microorganism (iii) of the genus Bifidobacterium species is the species Bifidobacterium longum.

Mention may in particular be made, as lysate capable of being used within the meaning of the invention, of the ingredient having the INCI name: Bifida Ferment Lysate.

Mention may in particular be made, by way of example, of the product sold under the name Repair Complex CLR® by K. Richter GmbH, and which is formed of an inactivated lysate of the species Bifidobacterium longum.

The total content, expressed as dry extract, of the lysate of the microorganism(s) (iii) of the genus Bifidobacterium species, present in the composition of the invention, preferably ranges from 0.001% to 20% by weight, more preferentially from 0.01% to 10% by weight, better still from 0.05% to 5% by weight, even better still from 0.1% to 2% by weight, with respect to the total weight of the composition.

### (iv) The Extracts of Yeast of the Genus Saccharomyces

The composition according to the present invention additionally comprises one or more extracts of yeast (iv) of the genus Saccharomyces.

The yeast(s) can be prepared by culturing in a conventional medium for the culturing of yeasts (for example: 10 g/l of yeast extract, 7 g/l of peptone digested by pepsin, 20 g/l of glucose and water in an amount sufficient to reach 1 litre).

The choice will preferentially be made of an aqueous yeast extract, that is to say a yeast extract which, subject to the unavoidable losses according to good manufacturing practices, contains all the water-soluble constituents of the yeast after lysis of the latter and removal by filtration of the membrane debris. This aqueous yeast extract will preferentially be redissolved.

Preferably, the aqueous extract of yeast of the genus Saccharomyces according to the present invention will be prepared by dissolving whole yeasts of the genus Saccharomyces in water (preferably distilled water). The suspension thus obtained will subsequently be subjected to hydrolysis of the proteins. Then the soluble and non-soluble phases of the solution obtained following this hydrolysis will be separated and the soluble phase will be recovered. This soluble phase will subsequently be subjected to sterilization.

In a preferential embodiment of the invention, the aqueous yeast extract thus obtained can subsequently be optionally dried and used in powder form. The extract, preferably in powder form, can also be placed in solution (the term used will then be yeast extract in solution), in particular in aqueous/alcoholic solution, preferably in aqueous/alcoholic solution, and more preferentially in aqueous/glycolic solution (for example a solution constituted of a mixture of water and pentylene glycol). The content of the aqueous yeast extract, present in this solution, preferably ranges from 0.5% to 8% by weight, more preferentially from 2% to 7% by weight, and better still from 3% to 5% by weight, with respect to the total weight of said solution.

The yeast extract used in the present invention preferably comprises water, the content of which is advantageously greater than or equal to 50% by weight, preferably greater than or equal to 60% by weight, more preferentially greater than or equal to 70% by weight, and better still greater than or equal to 80% by weight, with respect to the total weight of said extract.

Advantageously, the yeast extract in solution, which can be used according to the present invention, corresponds to the following characteristics:
a pH of between 5 and 8, preferably between 6 and 7, and/or
between 20 and 60 g of sugars per litre of solution (g/l), preferably between 20 and 50 g/l and more preferentially between 25 and 35 g/l, and/or
a concentration of solids (or dry matter) of between 10 and 60 g/l, preferably between 20 and 50 g/l and more preferentially between 37 and 47 g/l.

The population of whole yeasts used to prepare this aqueous yeast extract, that is to say before hydrolysis and/or sterilization, will preferentially be from 105 to 1010 colony-forming units per millilitre (or cfu/ml) of aqueous yeast solution.

The aqueous yeast extract which can be used in the composition of the invention preferably exhibits the following characteristics:
a total nitrogen content (according to the Kjeldahl method) ranging from 5% to 15% by weight, preferably from 6% to 12% by weight, and more preferentially from 7% to 10% by weight, with respect to the total weight of the yeast extract, and/or
a total content of free amino acids (according to the Sörensen method) ranging from 2% to 10% by weight, preferably from 3% to 7% by weight, and more preferentially from 4% to 6% by weight, with respect to the total weight of the yeast extract, and/or
a ratio by weight of the total content of assimilable amino nitrogen to the total nitrogen content, present in said extract, ranging from 0.4 to 0.7, preferably from 0.4 to 0.6, and more preferentially from 0.5 to 0.6.

The hydrolysis of the proteins is preferentially carried out by chemical or acidic hydrolysis or by the use of natural yeast enzymes. This hydrolysis is preferably carried out for at least 60%, more preferentially at least 80%, and better still at least 90% of all of the proteins present in the yeast solution after the lysis of the yeast.

The separation of the soluble and non-soluble phases obtained following the hydrolysis of the proteins can be carried out by any means known to a person skilled in the art depending on the nature of the desired extract. This separation of the phases can, for example, be carried out by filtration, decanting or centrifugation, filtration being the preferential means. Following this separation of the soluble and non-soluble phases, the soluble phase is recovered.

The sterilization can be carried out by any means known to a person skilled in the art and in particular by sterilizing filtration. The latter will be preferentially carried out by the use of a membrane filter, the size of the pores of which is chosen as a function of the size of the membrane elements which it is desired to remove, this technique being well known to a person skilled in the art.

Following this sterilization stage, the aqueous yeast extract obtained can be dried in order to provide it in powder form. This drying can also be carried out by any means known to a person skilled in the art, for example by evaporation, lyophilization or spray drying.

The yeast(s) of the genus Saccharomyces used in the present invention are preferably chosen from the following species: Saccharomyces bailii, Saccharomyces carlsbergensis, Saccharomyces uvarum, Saccharomyces cerevisiae, Saccharomyces delbrueckii, Saccharomyces exiguus, Saccharomyces fermentati, Saccharomyces florentinus, Saccharomyces fragilis, Saccharomyces fructuum, Saccharomyces heterogenicus, Saccharomyces oleaginosus, Saccharomyces rosei, Saccharomyces steineri, Saccharomyces boulardii, Saccharomyces kefir, Saccharomyces kluyveri and their mixtures.

More preferentially, the yeast of the genus Saccharomyces is a yeast of the species Saccharomyces cerevisiae.

Such compounds are in particular known under the INCI name: Yeast Extract.

Advantageously, the extract(s) of yeast of the genus Saccharomyces, present in the composition of the invention, do not comprise live yeast.

An example of extract of yeast of the genus Saccharomyces which can be used according to the present invention is sold by Silab under the name Firmalift® GRV. The CAS number of this extract is 8013-01-2 and its EINECS/ELINCS number is 232-387-9. This extract is provided in the form of a solution in a water/pentylene glycol mixture.

The total content (as dry matter or active material) of the extract(s) of yeast (iv) of the genus Saccharomyces, present in the composition according to the invention, preferably ranges from 0.001% to 5% by weight, more preferentially from 0.01% to 1% by weight, and better still from 0.02% to 0.5% by weight, with respect to the total weight of the composition.

### The Monosaccharides

The composition according to the present invention may optionally additionally comprise one or more monosaccharides.

The term “monosaccharides” is understood to mean, within the meaning of the present patent application, a monosaccharide sugar comprising at least 3 carbon atoms, preferably at least 5 carbon atoms, of formula Cₓ(H₂O)ₓ with x an integer greater than or equal to 3, preferably greater than or equal to 5; preferentially, x is greater than or equal to 6; in particular x is of between 5 and 7 inclusive; preferably x=6: they may be of D or L configuration, and of α or β anomerism, and also their salts and their solvates, such as the hydrates.

Preferably, the monosaccharide(s) are chosen from mannose, rhamnose, and also their α or β anomers, their optical isomers of L or D configuration, their solvates, such as the hydrates, and their mixtures.

More preferentially, the monosaccharide(s) are chosen from mannose, rhamnose and their mixtures, better still mannose.

Mannose is a monosaccharide (simple non-hydrolysable sugar) consisting of 6 carbon atoms; it is a hexose. Its empirical formula is C₆H₁₂O₆, the same as that of glucose, of which it is the C2 epimer (that is to say that its spatial configuration is strictly the same as that of glucose, except for the substituent of the 2 carbon, where it is inverted with respect to glucose). Mannose corresponds to the following formula (I), which also encompasses its enantiomers:

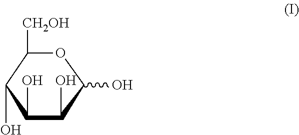

Mannose can also be provided in solvated form (including the hydrates) and in the form of a mixture of D and L stereoisomers referred to as DL-mannose.

Preferably, the monosaccharide is D-mannose of formula (II) below.

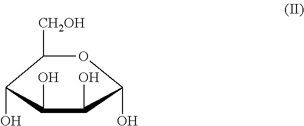

D-Mannose is naturally present in plants, in particular in certain fruits, including cranberries, or in hardwoods, such as beech and birch.

Mention may in particular be made, as example of D-mannose suitable for the invention, of the D-mannose sold by Danisco Sweeteners® or also Symrise®.

Rhamnose (or 6-deoxymannose) formally constitutes the product of deoxygenation of mannose at C₆. Rhamnose is found in nature in the L form. L-Rhamnose products are sold, for example, by Danisco Sweeteners® and also by Symrise.

Preferably, the monosaccharide(s) are chosen from mannose, and also its α or β anomers, its optical isomers of L or D configuration, its solvates and their mixtures. More preferentially, the monosaccharide is D-mannose.

The total content of the monosaccharide(s), when they are present in the composition of the invention, preferably ranges from 0.01% to 20% by weight, more preferentially from 0.05% to 10% by weight and better still from 0.1% to 5% by weight, with respect to the total weight of the composition.

Preferably, when the composition according to the invention comprises one or more monosaccharide(s), preferably chosen from mannose, rhamnose, and also their α or β anomers, their optical isomers of L or D configuration, their solvates, such as the hydrates, and their mixtures, the ratio by weight of the total content of the antioxidant(s) (i) to the total content of the monosaccharide(s), which are present in the composition of the invention, is greater than or equal to 1 and more preferentially ranges from 1 to 5.

Better still, when the composition comprises ascorbyl glucoside and mannose, the ratio by weight of the total content of ascorbyl glucoside to the total content of mannose, which are present in the composition of the invention, is preferably greater than or equal to 1 and more preferentially ranges from 1 to 5.

According to a preferred embodiment, the composition according to the invention comprises one or more monosaccharide(s), preferably chosen from mannose, rhamnose, and also their α or β anomers, their optical isomers of L or D configuration, their solvates, such as the hydrates, and their mixtures, better still from mannose, and also its α or β anomers, its optical isomers of L or D configuration, its solvates, such as the hydrates, and their mixtures.

### The Additional Saccharides

The composition according to the present invention can optionally also comprise one or more additional saccharides, different from the monosaccharides defined above, chosen from oligosaccharides, polysaccharides and their mixtures.

The term “polysaccharide” is understood to mean, within the meaning of the present invention, complex carbohydrates which are polymers constituted of several units n of monosaccharides, such as defined above, linked together by saccharide bonds, the number n being greater than or equal to 11, preferably of between 11 and 200, more preferentially between 11 and 100, and better still between 20 and 80. Mention may in particular be made, as example of polysaccharide, of inulin.

The term “oligosaccharides” is understood to mean, within the meaning of the present invention, oligomers constituted of several units n of monosaccharides, such as defined above, via one or more α or β glycosidic bonds, the number n being of between 2 and 10, preferably between 2 and 6. Mention may in particular be made, as examples of oligosaccharides, of fructooligosaccharides, glucooligosaccharides, oligosaccharides derived from soya, pyrodextrins, isomaltooligosaccharides, xylooligosaccharides and transgalactooligosaccharides.

Preferably, the additional saccharide(s) are chosen from inulin, fructooligosaccharide, glucooligosaccharide, oligosaccharides derived from soya, pyrodextrins, isomaltooligosaccharide, xylooligosaccharide, transgalactooligosaccharide and their mixtures.

The oligosaccharides and the polysaccharides which can be used according to the present invention are carbohydrates.

The oligosaccharides and the polysaccharides which can be used can be produced in particular from glucose, galactose, xylose, maltose, sucrose, lactose, starch, xylan, hemicellulose, inulin, gums, in particular acacia gum, or a mixture of these.

Mention will be made, by way of example, of the following oligosaccharides and/or polysaccharides:
inulin

Inulin is particularly abundant in the rhizomes of plants, in particular Jerusalem artichoke and endive, from which it is extracted industrially. It is also found in other plants belonging to the family of the Asteraceae, such as dahlia onions and burdock. It is regarded as a soluble dietary fibre.

Inulins are polydispersed linear polymers of general formula (III) (also represented below): GFn, in which G is a glucose unit, F is a fructose unit and n varies from 2 to 60, indeed even to more than 60, the fructose units being linked together by a β (2→1) bond. Inulins therefore correspond to a chain of fructose units terminated by a glucose unit.

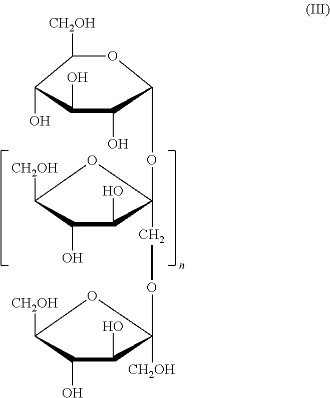

Mention may in particular be made, among the inulins which can be used and which are commercially available, of Inutec H25P (with n of between 2 and 7) and Inutec N25 from Orafti (with mean n=25).
fructooligosaccharides

Fructooligosaccharides (or FOSs), also called oligofructoses or oligofructans, are short chains of fructose connected together by β-1,2 bonds. Fructooligosaccharides (or FOSs) correspond to the general formula: G(F)n, where G is a glucose unit, F is a fructose unit et n varies from 1 to 10.

Fructooligosaccharides (FOSs) are produced:
by partial enzymatic hydrolysis of inulin (e.g.: Raftilose® from Orafti-Belgique),
by enzymatic synthesis starting from sucrose (e.g.: Actilight® from Beghin Meiji Industries-France), or
by extraction from yacon or jicama (Polymnia sonchifolia, synonym: Smallanthus sonchifolia); in particular, the extraction is carried out in the absence of solvent, by cold pressing of the yacon tubers.

The fructooligosaccharide (FOS) which can be used in the present invention is preferably a FOS mixture. Mention may in particular be made of GF2+GF3+GF4 mixtures, such as Quantom FOS95 from Quantum Hi-Tech or Actilight® from Beghin Meiji Industries-France, the latter corresponding to a mixture of 37% GF2, 53% GF3 and 10% GF4.
Glucooligosaccharides

Glucooligosaccharides (GOSs), or oligoglucans, are oligosaccharides constituted of a sequence of α-1,6-linked glucose units which can also contain α-1,2; α-1,3; or α-1,4 bonds. They are synthesized by a transglucosylation reaction catalysed by enzymes of the family of the glucansucrases.

Preferably, the glucooligosaccharides are oligosaccharides constituted of a sequence of α-1,6- and α-1,2-linked glucose units.

Advantageously, the number of glucose units is of between 2 and 10, preferably between 4 and 6, and more preferentially the number of glucose units is 4.

In addition, the glucooligosaccharides can be synthesized by the polymerization of glucose molecules, a reaction catalysed by a specific enzyme of glycosyltransferase type, extracted and purified from a bacterial strain of Leuconostoc mesenteroides. This reaction requires the use of an acceptor: maltose (Glucose-Glucose) but also of a donor of glucose: sucrose (Glucose-Fructose).

In a preferred embodiment, the glucooligosaccharides (GOSs) have the formula (V) below:

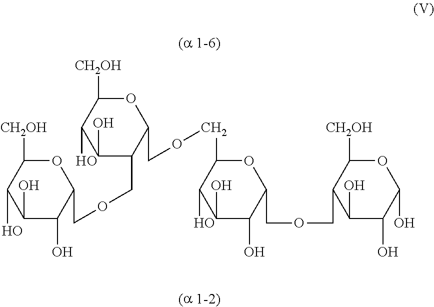

Mention may be made, among the GOSs which can be used and which are commercially available, of the product Bioecolia® from Solabia.
Oligosaccharides derived from soya

These oligosaccharides are extracted directly from the soya bean and do not require any enzymatic treatment. They naturally contain raffinose and stachyose, the formula of which is as follows:

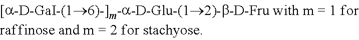

Mention may in particular be made, among the oligosaccharides which can be used, of Soya-oligo from Calpis Food Ind. Japan.
Pyrodextrins

Pyrodextrins are a mixture of oligosaccharides originating from the hydrolysis of starch.
Isomaltooligosaccharides

Isomaltooligosaccharides are produced from starch. These are α-(1,6)-linked glucose oligomers, the degree of polymerization of which is of between 2 and 5. Use may be made, by way of example, of Isomalto900P from Showa Sango.
Xylooligosaccharides

Xylooligosaccharides are oligosaccharides constituted of xylose units linked together by β-(1,4) bonds. Use may be made, by way of example, of Xylo 95P from Suntory Limited.
Transgalactooligosaccharides

Transgalactooligosaccharides (or TOSs) are linear oligomers of galactose, of chemical structure α-D-glucose-(1→4)-[β-D-galactose-(1→6)-]ₙ (with n of between 2 and 5) obtained by fermentation of lactose.

Use may be made, by way of example, of TOS 100 from Yakult Honsha Co. Ltd Japan.

The total content of the additional saccharide(s), different from the monosaccharides, chosen from oligosaccharides, polysaccharides and their mixtures, when they are present in the composition of the invention, preferably ranges from 0.01% to 20% by weight, more preferentially from 0.05% to 20% by weight, better still from 0.05% to 10% by weight, and more preferentially still from 0.05% to 5% by weight, indeed even from 0.1% to 2% by weight, with respect to the total weight of the composition.

According to a preferred embodiment, the composition according to the invention comprises one or more additional saccharides, different from the monosaccharides, chosen from oligosaccharides, polysaccharides and their mixtures.

According to a particularly preferred embodiment of the invention, the composition comprises a mixture of at least one oligosaccharide and/or of at least one polysaccharide. In other words, the composition can comprise a mixture of at least two oligosaccharides, a mixture of at least two polysaccharides or a mixture of at least one oligosaccharide and of at least one polysaccharide.

According to a first alternative form of this embodiment, the composition comprises a mixture of at least two oligosaccharides of the same type. For example, as mentioned above, use may be made of a mixture of FOSs, in particular a mixture of GF2+GF3+GF4, such as Quantom FOS95 from Quantum Hi-Tech or Actilight® from Beghin Meiji Industries-France, the latter corresponding to a mixture of 37% GF2, 53% GF3 and 10% GF4.

According to a second alternative form of this embodiment, the composition comprises a mixture of oligosaccharides and/or of polysaccharides of different types. The invention relates in particular to the use of a mixture of inulin with a GOS, an FOS, oligosaccharides derived from soya, pyrodextrins, an isomaltooligosaccharide, a xylooligosaccharide and/or a transgalactooligosaccharide. Preferably, use is made of a mixture of inulin and of GOS, such as Bioline from Gova Ingredients. The composition can also comprise a mixture of a GOS with a FOS.

Preferably, according to this second alternative form of the embodiment, the composition according to the invention comprises a mixture of oligosaccharides comprising:
at least one glucooligosaccharide (GOS), and
at least one fructooligosaccharide (FOS),

the total content of the glucooligosaccharide(s) ranging from 0.01% to 10% by weight, preferably from 0.05% to 10% by weight, more preferentially from 0.05% to 5% by weight, better still from 0.1% to 1% by weight, and more preferentially still from 0.2% to 0.8% by weight, with respect to the total weight of the composition;

the total content of the fructooligosaccharide(s) (FOSs) ranging from 0.001% to 5% by weight, preferably from 0.01% to 0.02% by weight, more preferentially from 0.01% to 1% by weight, better still from 0.01% to 0.5% by weight, and more preferentially still from 0.05% to 0.3% by weight, with respect to the total weight of the composition.

Advantageously, in this second alternative form of the specific embodiment of the invention, the composition according to the invention comprises a mixture of oligosaccharides comprising:
at least one glucooligosaccharide (GOS), and
at least one fructooligosaccharide (FOS),

the ratio by weight of the total content of glucooligosaccharides to the total content of fructooligosaccharides ([GOS/FOS] ratio) is greater than or equal to 2. Preferably, this ratio by weight ranges from 2 to 4 and more preferentially from 3 to 4.

Use may in particular be made of the mixture of prebiotic oligosaccharides and of probiotic microorganisms sold under the name Ecoskin® by Solabia. Said mixture comprises in particular:
between 60% and 80% by weight of at least one glucooligosaccharide (GOS), with respect to the total weight of the mixture, and
between 10% and 25% by weight of at least one fructooligosaccharide (FOS), with respect to the total weight of the mixture.

In particular, said mixture comprises 70% by weight of glucooligosaccharide (GOS), 19% by weight of Polymnia sonchifolia tuber juice, 1% by weight of Lactobacillus acidophilus and Lactobacillus casei, and 10% by weight of maltodextrin.

### The Probiotic Microorganisms

The composition according to the present invention can optionally additionally comprise one or more probiotic microorganisms, different from the microorganisms (iii) of the genus Bifidobacterium species defined above.

The term “probiotic microorganism” is understood to mean, within the meaning of the present invention, a live microorganism which, when it is consumed in a suitable amount, has a positive effect on the health of its host (Joint FAO/WHO Expert Consultation on Evaluation of Health and Nutritional Properties of Probiotic in Food Including Powder Milk with Live Lactic Acid Bacteria, 6 Oct. 2001) and which can in particular improve the intestinal microbial balance.

In the case of keratin materials, in particular human keratin materials, such as the skin or the scalp, said probiotic microorganism is a probiotic microorganism which, when it is applied to the keratin material in a suitable amount, has a positive effect on the aesthetic qualities of said keratin material.

Preferably, the probiotic microorganism(s) which can be used according to the present invention are chosen from probiotic microorganisms from the group of the lactic acid bacteria, such as in particular the Lactobacillus species, and their mixtures. Mention may be made, by way of illustration of these lactic acid bacteria, of Lactobacillus casei, Lactobacillus acidophilus and their mixtures.

Advantageously, the probiotic microorganism(s) are chosen from Lactobacillus acidophilus, Lactobacillus alimentarius, Lactobacillus curvatus, Lactobacillus delbruckii subsp. lactis, Lactobacillus gasseri, Lactobacillus johnsonii, Lactobacillus reuteri, Lactobacillus casei, Lactobacillus rhamnosus (Lactobacillus GG), Lactobacillus sake, Lactobacillus lactis, Streptococcus thermophilus, Lactobacillus delbrueckii, Lactobacillus helveticus, Lactobacillus salivarius, Lactobacillus plantarum, Lactobacillus sakei, Lactobacillus brevis, Lactobacillus buchneri, Lactobacillus fermentum, Lactobacillus bulgaricus, Lactobacillus longum and their mixtures.

The probiotic microorganism(s) can be included in the composition according to the invention in a live, semi-active or inactivated (dead) form. They can also be included in the form of fractions of cell components or in the form of metabolites. The probiotic microorganism(s) or metabolite(s) or fraction(s) can also be introduced in the form of a freeze-dried powder, of a culture supernatant and/or, if appropriate, in a concentrated form.

According to a preferential embodiment of the invention, these microorganisms are in inactivated form.

The terms “in inactivated form”, “in non-revivable form” and “in dead form” are synonymous here. Bacteria “in semi-active form” are bacteria which have partially or totally lost their pathogenic properties, if any.

The total content of the probiotic microorganism(s), different from the microorganisms (iii), when they are present in the composition according to the invention, advantageously ranges from 0.0001% to 20% by weight, preferably from 0.0001% to 10% by weight, more preferentially from 0.001% to 5% by weight, and better still from 0.001% to 1% by weight, with respect to the total weight of the composition.

This or these microorganism(s) can be included in the compositions according to the invention in a live, semi-live or inactivated (dead) form, preferably inactivated for example by heat or by high pressure.

In the case where the microorganisms are formulated in a composition in live form, the amount of live microorganisms can preferably range from 103 to 1015 cfu/g, more preferentially from 105 to 1015 cfu/g and better still from 107 to 1012 cfu/g of microorganisms per gram of composition.

In a particularly preferred embodiment, the composition according to the present invention comprises a mixture of oligosaccharides comprising:
one or more glucooligosaccharides (GOSs), and
one or more fructooligosaccharides (FOSs),

the total content of the glucooligosaccharide(s) ranging from 0.01% to 10% by weight, preferably from 0.05% to 10% by weight, more preferentially from 0.05% to 5% by weight, better still from 0.1% to 1% by weight, and more preferentially still from 0.2% to 0.8% by weight, with respect to the total weight of the composition;

the total content of the fructooligosaccharide(s) ranging from 0.001% to 5% by weight, preferably from 0.01% to 0.01% by weight, more preferentially from 0.01% to 1% by weight, better still from 0.01% to 0.5% by weight, and more preferentially still from 0.05% to 0.3% by weight, with respect to the total weight of the composition.

According to this embodiment, the composition invention preferably additionally comprises one or more probiotic microorganisms chosen from the bacteria of the genus Lactobacillus, more preferentially from Lactobacillus casei, Lactobacillus acidophilus and their mixtures; the total content of the probiotic microorganism(s) advantageously ranging from 0.0001% to 10% by weight, preferably from 0.001% to 5% by weight, and more preferentially from 0.001% to 1% by weight, with respect to the total weight of the composition.

Advantageously, the composition according to the invention comprises a mixture of oligosaccharides comprising:
one or more glucooligosaccharides (GOSs), and
one or more fructooligosaccharides (FOSs),

the ratio by weight of the total content of glucooligosaccharides to the total content of fructooligosaccharides ([GOS/FOS] ratio) being greater than or equal to 2. Preferably, this ratio by weight ranges from 2 to 4 and more preferentially from 3 to 4.

Use can in particular be made, by way of example, of the mixture of prebiotic oligosaccharides and of probiotic microorganisms sold under the name Ecoskin® by Solabia. Said mixture comprises in particular:
between 60% and 80% by weight of at least one glucooligosaccharide (GOS), with respect to the total weight of the mixture,
between 10% and 25% by weight of at least one fructooligosaccharide (FOS), with respect to the total weight of the mixture, and
between 0.001% and 15% by weight of at least one probiotic microorganism, in particular chosen from Lactobacillus casei, Lactobacillus acidophilus and their mixtures, with respect to the total weight of the mixture.

In particular, said mixture comprises 70% by weight of glucooligosaccharide (GOS), 19% by weight of Polymnia sonchifolia tuber juice, 1% by weight of Lactobacillus acidophilus and Lactobacillus casei, and 10% by weight of maltodextrin.

### The Non-Ionic Surfactants

The composition according to the present invention can also optionally comprise one or more non-ionic surfactants, different from the oxyalkylenated esters of fatty acid and of sorbitan (ii) defined above.

These surfactants can in particular contribute to retaining the clarity or transparency of the composition.

Examples of non-ionic surfactants which can be used in the compositions of the present invention are described, for example, in the Handbook of Surfactants by M. R. Porter, published by Blackie & Son (Glasgow and London), 1991, pages 116-178. They are chosen in particular from alcohols, α-diols, (C₁-C₂₀)alkylphenols or acids which are fatty, these compounds being polyethoxylated, polypropoxylated or polyglycerolated and having at least one fatty chain comprising, for example, from 8 to 18 carbon atoms, it being possible for the number of ethylene oxide or propylene oxide groups to range in particular from 1 to 100 and it being possible for the number of glycerol groups to range in particular from 1 to 30.

Mention may be made, as examples of non-ionic surfactants, different from the oxyalkylenated esters of fatty acid and of sorbitol (ii), which can be used according to the present invention, of the following non-ionic surfactants:
oxyalkylenated (C₈-C₂₄)alkylphenols;
saturated or unsaturated, linear or branched, oxyalkylenated or glycerolated C₈ to C₄₀ alcohols;
saturated or unsaturated, linear or branched, oxyalkylenated C₈ to C₃₀ fatty acid amides;
esters of saturated or unsaturated, linear or branched, C₈ to C₃₀ acids and of polyols or of polyethylene glycols;
esters of fatty acids and of sucrose;
(C₈-C₃₀)alkyl(poly)glucosides, (C₈-C₃₀)alkenyl(poly)glucosides, which are optionally oxyalkylenated (0 to 10 oxyalkylene units) and which comprise from 1 to 15 glucose units, esters of (C₈-C₃₀)alkyl(poly)glucosides;
saturated or unsaturated oxyethylenated vegetable oils;
condensates of ethylene oxide and/or of propylene oxide;
N—(C₈-C₃₀)alkylglucamine and N—(C₈-C₃₀)acylmethylglucamine derivatives;
aldobionamides;
amine oxides;
oxyethylenated and/or oxypropylenated silicones;
and their mixtures.

The oxyalkylene units are more particularly oxyethylene or oxypropylene units, or their combination, preferably oxyethylene units.

The number of moles of ethylene oxide and/or of propylene oxide preferably ranges from 1 to 250, more particularly from 2 to 100 and better still from 2 to 50; the number of moles of glycerol ranges in particular from 1 to 50 and better still from 1 to 10.

Advantageously, the non-ionic surfactants, different from the oxyalkylenated esters of fatty acid and of sorbitol (ii) according to the invention, do not comprise oxypropylene units.

Use is preferably made, as examples of glycerolated non-ionic surfactants, of mono- or polyglycerolated C₈ to C₄₀ alcohols, comprising from 1 to 50 mol of glycerol, preferably from 1 to 10 mol of glycerol.

Among the glycerolated alcohols, it is more particularly preferred to use the C₈/C₁₀ alcohol having 1 mol of glycerol, the C₁₀/C₁₂ alcohol having 1 mol of glycerol and the C₁₂ alcohol having 1.5 mol of glycerol.

The non-ionic surfactant(s), different from the oxyalkylenated esters of fatty acid and of sorbitol (ii), which can be used in the composition according to the invention are preferentially chosen from:
oxyethylenated C₈ to C₄₀ alcohols comprising from 1 to 100 mol of ethylene oxide, preferably from 2 to 50 and more particularly from 2 to 40 mol of ethylene oxide;
saturated or unsaturated oxyethylenated vegetable oils comprising from 1 to 100 mol of ethylene oxide, preferably from 2 to 50;
(C₈-C₃₀)alkyl(poly)glucosides, which are optionally oxyalkylenated (0 to 10 EO) and which comprise from 1 to 15 glucose units;
mono- or polyglycerolated C₈ to C₄₀ alcohols, comprising from 1 to 50 mol of glycerol, preferably from 1 to 10 mol of glycerol;
saturated or unsaturated, linear or branched, oxyalkylenated C₈ to C₃₀ fatty acid amides;
esters of saturated or unsaturated, linear or branched, C₈ to C₃₀ acids and of polyols or of polyethylene glycols;
and their mixtures.

Preferably, the non-ionic surfactant(s), different from the oxyalkylenated esters of fatty acid and of sorbitol (ii), are chosen from saturated or unsaturated, linear or branched, oxyalkylenated or glycerolated, preferentially oxyethylenated, C₈ to C₄₀ alcohols; saturated or unsaturated oxyethylenated vegetable oils comprising from 1 to 100 mol of ethylene oxide, preferably from 2 to 50, and their mixtures.

The total content of the non-ionic surfactant(s), different from the oxyalkylenated esters of fatty acid and of sorbitol (ii), when they are present in the composition of the invention, preferably ranges from 0.05% to 10% by weight, more preferentially from 0.1% to 5% by weight, better still from 0.2% to 3% by weight, and even better still from 0.3% to 2% by weight, with respect to the total weight of the composition.

The composition according to the invention preferably comprises water. Advantageously, the total water content ranges from 50% to 98% by weight, preferably from 60% to 95% by weight, preferentially from 70% to 90% by weight, with respect to the total weight of the composition.

The composition according to the invention can advantageously additionally comprise one or more linear or branched monoalcohols, having from 1 to 6 carbon atoms, and more preferentially from 1 to 4 carbon atoms, in particular chosen from ethanol, propanol, butanol, isopropanol, isobutanol and their mixtures, better still ethanol.

The monoalcohols capable of being used according to the invention are linear or branched; they are thus not cyclic or aromatic.

Said monoalcohol(s) may be present in the composition in a total content ranging from 5% to 40% by weight, preferably from 10% to 35% by weight, more preferentially from 12% to 30% by weight, and better still from 15% to 25% by weight, with respect to the total weight of the composition.

The composition according to the invention can optionally also comprise one or more organic solvents different from the monoalcohols above, and which can be chosen from polyols, polyethylene glycols, aromatic alcohols and their mixtures.

Mention may in particular be made, as examples of organic solvents different from the monoalcohols described above which can be used according to the invention, of propylene glycol, dipropylene glycol, isoprene glycol, butylene glycol, pentylene glycol, glycerol, sorbitol, benzyl alcohol, phenoxyethanol, and their mixtures.

The total content of the organic solvent(s) different from the monoalcohols described above, when they are present in the composition of the invention, preferably ranges from 0.1% to 20% by weight, more preferentially from 0.5% to 10% by weight, and better still from 1% to 5% by weight, with respect to the total weight of the composition.

When the composition according to the invention comprises one or more organic solvents, in particular chosen from linear or branched monoalcohols having from 1 to 6 carbon atoms, the composition is an aqueous/alcoholic composition. In a preferred embodiment, the composition according to the invention comprises one or more linear or branched monoalcohols having from 1 to 6 carbon atoms.

The composition according to the present invention can also optionally comprise one or more additional compounds different from the compounds defined above, preferably chosen from cationic, anionic and amphoteric surfactants, cationic, anionic, non-ionic and amphoteric polymers, thickening agents, penetrating agents, sequestering agents, fragrances, buffers, dispersing agents, film-forming agents, ceramides, preserving agents, opacifying agents, lubricants (or anticaking agents) and their mixtures.

Preferably, when the above additional compound(s) are present in the composition according to the invention, the additional compound(s) are generally present in a content, for each of them, of between 0.01% and 20% by weight, with respect to the weight of the composition.

In an advantageous embodiment, the composition according to the invention can comprise one or more antidandruff agents and/or one or more agents for combating hair loss.

Of course, a person skilled in the art will take care to choose this or these optional additional compound(s) such that the advantageous properties intrinsically attached to the composition of the invention are not, or not substantially, detrimentally affected by the envisaged addition(s).

The optional thickening agent(s) can be chosen from inorganic or organic thickening agents. The thickening agent can be non-ionic, cationic, anionic or amphoteric.

Mention may in particular be made, as thickening agents which can be used according to the present invention, of carboxyvinyl polymers, such as crosslinked acrylic and/or methacrylic acid polymers, in particular crosslinked acrylic acid homopolymers (carbomer), such as those sold under the name Carbopol by Goodrich, polyacrylates and polymethacrylates, such as the products sold under the names Lubrajel or Norgel by Guardian or under the name Hispagel by Hispano Chimica; polyacrylamides, such as the product sold under the name Sepigel 305 by SEPPIC; polysaccharides, such as alginates, cellulose and its derivatives, in particular carboxymethylcellulose, hydroxymethylcellulose, hydroxypropylcellulose and microcrystalline cellulose; natural gums, such as xanthan gum, guar gum, locust bean gum, acacia gum, scleroglucans, chitin and chitosan derivatives, carrageenans; or clays, such as montmorillonite and derivatives, bentones and derivatives, or magnesium aluminium silicates and derivatives (Veegum).

According to a specific embodiment of the invention, the composition comprises at least one thickening agent chosen from crosslinked acrylic and/or methacrylic acid polymers.

According to a specific embodiment of the invention, the composition comprises at least one thickening agent chosen from crosslinked acrylic acid homopolymers.

The total content of the thickening agent(s), when they are present in the composition of the invention, preferably ranges from 0.05% to 5% by weight and more preferentially from 0.1% to 3% by weight, with respect to the weight of the composition.

The pH of the composition according to the invention, when it is aqueous, advantageously varies from 5 to 9, preferably from 6 to 8, more preferentially from 6.5 to 7.5.

The pH of the composition can be adjusted to the desired value by means of basifying agents or acidifying agents customarily used. Mention may be made, among the basifying agents, by way of examples, of aqueous ammonia, alkanolamines or inorganic or organic hydroxides. Mention may be made, among the acidifying agents, by way of examples, of inorganic or organic acids, such as hydrochloric acid, orthophosphoric acid, sulfuric acid, carboxylic acids, such as, for example, acetic acid, tartaric acid, citric acid or lactic acid, or sulfonic acids.

The composition according to the present invention is preferably applied topically, in particular on the scalp and/or the hair.

The compositions intended for administration by the external topical route can be solutions or dispersions of the lotion or serum type, of the cream type, or aqueous gels. These compositions are prepared according to the usual methods.

Preferably, the composition by the topical route according to the invention can advantageously be formulated in any presentation form suitable for hair care and/or care of the scalp, in particular in the form of a hair lotion, of a shampoo, in particular an anti-dandruff shampoo, of a conditioner, of a disentangler, of a hair cream or gel, of a treating lotion, of a lotion or gel for combating hair loss, of an anti-parasitic shampoo, of a treating shampoo, in particular an anti-seborrhoeic shampoo, or of a product for care of the scalp, in particular an anti-irritant product, restructuring product.

The composition according to the invention is advantageously provided in a thickened, indeed even gelled, form, in particular in the form of a thickened, indeed even gelled, lotion or of a thickened, indeed even gelled, serum.

The composition according to the invention is advantageously provided in the form of a clear to transparent fluid, preferably a transparent fluid.

The transparency of the composition according to the invention can be characterized by the measurement of its turbidity, by turbidimetry (in NTU units). In the context of the present invention, the turbidity measurements were carried out using a UV-Vis Cary 100 model UV spectrophotometer sold by Agilent. It is also possible to measure the turbidity of the composition using a turbidimeter, such as the HI 88713-ISO model from Hanna Instruments.

Advantageously, the turbidity of the composition according to the invention, measured at ambient temperature (25° C.) and atmospheric pressure, is less than or equal to 250 NTU units, preferably less than or equal to 200 NTU units, more preferentially less than or equal to 100 NTU units, better still less than or equal to 50 NTU units, more preferentially still less than or equal to 20 NTU units, indeed even less than or equal to 10 NTU units.

Preferentially, the composition according to the invention comprises:
one or more oxyalkylenated esters of fatty acids and of sorbitan,
one or more antioxidants chosen from ascorbic acid and its derivatives,
one or more microorganisms of the genus Bifidobacterium species, one of its fractions, one of its metabolites or their mixtures, the microorganism(s) being in the lysate form,
one or more extracts of yeast of the genus Saccharomyces,
one or more monosaccharides,
optionally one or more additional saccharides, chosen from oligosaccharides, polysaccharides and their mixtures,
water, and
optionally one or more linear or branched monoalcohols having from 1 to 6 carbon atoms.

Better still, the composition according to the invention comprises:
one or more esters of saturated or unsaturated, linear or branched, C₈ to C₃₀ fatty acids and of sorbitan, comprising from 1 to 20 oxyethylene units, and their mixtures,
one or more antioxidants chosen from ascorbic acid, its derivatives and their mixtures, and more preferentially the antioxidant is ascorbyl glucoside,
one or more microorganisms of the genus Bifidobacterium species, one of its fractions, one of its metabolites or their mixtures, the microorganism(s) being in the lysate form,
one or more extracts of yeast of the genus Saccharomyces,
one or more monosaccharides preferably chosen from mannose, and also its α or β anomers, its optical isomers of L or D configuration, its solvates and their mixtures,
one or more additional saccharides chosen from oligosaccharides, polysaccharides and their mixtures,
water, and
one or more linear or branched monoalcohols having from 1 to 6 carbon atoms.

Another subject-matter of the present invention is a method for the cosmetic treatment of keratin materials, in particular human keratin materials, such as the scalp and the hair, comprising the application, to said keratin materials, of a composition as defined above.

Optionally, the application of the composition may or may not be followed by a rinsing operation, after an optional leave-on time.

Preferably, the application of the composition according to the invention is not followed by a rinsing operation.

Preferably, the cosmetic treatment method is a hair care method.

The present invention additionally relates to the non-therapeutic cosmetic use of a composition as defined above for maintaining, indeed even restoring, the balance of the microbial flora of the scalp.

The invention additionally relates to a device comprising one or more containers which comprises (comprise) the composition according to the invention as defined above.

The device can additionally comprise at least one dispensing member which makes it possible to dispense the composition, which can, for example, be a dispensing orifice or an applicator.

The dispensing organ can be adjoining said container (in this case, the device will be constituted only of a single item); the device can also be made of several parts, for example the container, on the one hand, and the dispensing organ, on the other hand.

The device can thus comprise at least one dispensing orifice making it possible to dispense the composition. The dispensing orifice can be closed by a closing member.

The device can thus comprise an applicator for facilitating the application of the composition. The applicator can, for example, be a comb, a fine brush or a coarse brush, indeed even a pipette or a felt, or also a dropper.

Advantageously, the composition is applied directly using the device via the dispensing orifice, optionally comprising an applicator. In other words, the composition is not taken up in the device. Thus, the device of the invention makes it possible to facilitate the application of the composition and to obtain an improved rate of restitution of said composition.

Advantageously, the application and the dispensing of the composition do not require a pump or a propellant. The composition flows by gravity via the dispensing orifice. A pressure can optionally be applied to the container to facilitate the flow and the dispensing of the ready-to-use composition.

The device can advantageously be a dropper optionally combined with a bottle, a pump-action spray, a bottle, a pipette, for example a single-dose pipette, and the like.

The following examples serve to illustrate the invention without, however, exhibiting a limiting nature.

### EXAMPLES

### 1. Example 1

Compositions A and B according to the invention were prepared from the ingredients, the contents of which are indicated in the table below (% by weight of active material, unless otherwise indicated).

TABLE 1

| | Composition A: | Composition B: |
|---|---|---|
| | Concentration | Concentration |
| | (% by weight) | (% by weight) |
| Carbomer | 0.25 | 0.25 |
| Polysorbate 21 | 1 | 1 |
| Mannose | 0.5 | 0.5 |
| α-Glucan | 0.3% of | 0.3% of |
| oligosaccharide (and) | commercial | commercial |
| Polymnia sonchifolia | product | product |
| tuber juice (and) | | |
| Lactobacillus (1) | | |
| Yeast extract (2) | 1% of | 1% of |
| | commercial | commercial |
| | product | product |
| Bifida Ferment | 10% of | 10% of |
| Lysate (3) | commercial | commercial |
| | product | product |
| Ascorbyl glucoside | 1 | 1 |
| Aminomethylpropanol | 0.43 | 0.43 |
| Fragrance | 0.6 | 0.6 |
| PEG-40 hydrogenated | 0.5 | 0.5 |
| castor oil | | |
| Ethanol | 20 | — |
| Water | q.s. 100 | q.s. 100 |

(1) Ecoskin RS (Solabia)
(2) Firmalift GRV (Solabia): Extract of yeast Saccharomyces cerevisiae in a water/pentylene glycol mixture
(3) Repair Complex CLR PF (CLR)

The compositions obtained are clear; they can be applied to the scalp and the hair and then be left to dry in the open air, without a rinsing stage after application. The compositions have a pH of approximately 7.

After use of these compositions daily for 21 days, a significant decrease in the feelings of discomfort of the scalp (stinging, feeling of heating) and in the frequency of scratching was observed.

The compositions do not leave the hair and/or the roots greasy; on the contrary, the hair is clean, without an effect of the fibres sticking together.

41 volunteers were asked to self-evaluate the effectiveness of the product on the hair and scalp at TO (immediately after application) and at T21.

The following results are obtained:
Immediately after application:
the product provides the scalp with a refreshing feeling,
the scalp is hydrated,
the feelings of heating, of itching and/or of stinging are weakened:
After 21 days of application:
the product provides the scalp with a refreshing feeling,
the scalp is comfortable, hydrated, less sensitive and soothed,
the feelings of heating, of itching and/or of stinging are weakened,
the product provides a feeling of well-being; its texture is pleasant.

### 2. Example 2

A study on 42 volunteers, composed of men and women between 18 and 60 years having a sensitive scalp, was carried out.

The volunteers applied 4.5 ml of Composition A of Example 1 over the whole of the scalp, at home every day for 21 days (from DI to D21), under the normal conditions of use, to dry hair, according to the following protocol:
three lines are defined from the forehead to the nape of the neck: one on the right-hand side, a central one and one on the left-hand side,
4.5 ml of serum are applied, the product being deposited by touch and by drop on these three lines as homogeneously as possible,
the product is distributed over the whole of the scalp using circular movements with the pads of the fingers.

At D0 (before the start of the application) and at D21 (after 21 days of application), the following measurements were carried out:
(a) Evaluation of the effect on the biochemical markers by the method of withdrawal of scrubbings (amount [SQOOH] by LC/MS analysis) on a delimited region of the scalp:

The evaluation of the oxidation of squalene, known to be an irritant, is measured between D0 and D21.

The following results are obtained (amount of SQOOH, in ng/mg of proteins):

TABLE 2

| | Mean | |
|---|---|---|
| | (over 42 people) | p-value |
| (Measurement | −23.3 | 0.0174 |
| at D 21) − | | |
| (measurement | | |
| at D 0) | | |

A significant decrease in the evaluation of the oxidation of squalene is observed.
(b) Evaluation of the effect of the composition on the cutaneous barrier:

This evaluation based on instrumental measurements of the level of TEWL (transepidermal water loss) of the skin of the scalp, carried out on a mini surface area of 2.25 cm², i.e. a square with a side length of 1.5 cm, with a Vapometer® with maintenance of the imperceptible water loss (up to 6 exact measurements per experimental time):

The following results are obtained (measurement of TEWL, expressed in g·m⁻²·h⁻¹):

TABLE 3

| | Mean | Standard |
|---|---|---|
| | (over 42 people) | deviation |
| Measurement | 14.28 | 4.31 |
| at D 0 | | |
| Measurement | 14.9 | 4.96 |
| at D 21 | | |
| (Measurement | 0.62 | 0.66 |
| at D 21) − | | |
| (measurement | | |
| at D 0) | | |

A significant difference in the barrier function of the scalp between D0 and D21 is not observed: the barrier function is retained (is not detrimentally affected) by the use of the composition according to the invention.
(c) Evaluation of the effect of the composition on reducing the discomforts of the scalp (itching, stinging, heating):

Each factor of the discomfort was evaluated on a scale from 0 to 9 by the 42 volunteers at D0 (before application), D0 imm (immediately: just after the 1st application of the product) and at D21.

The grade of 9 corresponds to a high level of discomfort, the grade of 0 corresponds to a zero level of discomfort, with regard to the factor evaluated.

The following results are obtained:

TABLE 4

| | Comparison | Mean | p-value |
|---|---|---|---|
| Itching | D 0 imm − D 0 | −2.17 | <0.0001 |
| | D 21 − D 0 | −4.25 | <0.0001 |
| Stinging | D 0 imm − D 0 | −1.62 | <0.0001 |
| | D 21 − D 0 | −3.33 | <0.0001 |
| Heating | D 0 imm − D 0 | −2.25 | <0.0001 |
| | D 21 − D 0 | −2.89 | <0.0001 |
| Overall | D 0 imm − D 0 | −6.04 | |
| discomfort | D 21 − D 0 | −10.48 | |

A significant decrease in the discomfort of the scalp is observed from the first application, and a substantial decrease is observed after three weeks of application.

### 3. Example 3

Compositions C (according to the invention) and C′ (comparative) were prepared from the ingredients, the contents of which are indicated in the tables below (% by weight of active material (AM), unless otherwise indicated):

TABLE 5

| | Composition C | Composition C′ |
|---|---|---|
| | (invention) | (comparative) |
| Carbomer | 0.5 | 0.5 |
| Polysorbate 21 | 0.5 | 0.5 |
| Mannose | 0.5 | 0.5 |
| α-Glucan | 0.3% of | 0.3% of |
| oligosaccharide (and) | commercial | commercial |
| Polymnia sonchifolia | product | product |
| tuber juice (and) | | |
| Lactobacillus (1) | | |
| Yeast extract (Faex | 0.042 AM | 0.042 AM |
| extract) (2) | (1% of | (1% of |
| | commercial | commercial |
| | product) | product) |
| Bifida Ferment | 9.86 AM | 9.86 AM |
| Lysate (3) | (10% of | (10% of |
| | commercial | commercial |
| | product) | product) |
| Ascorbyl glucoside | 1 | — |
| Tocopherol (Vitamin | — | 1 |
| E) | | |
| Aminomethylpropanol | 0.43 | 0.43 |
| Fragrance | 0.6 | 0.6 |
| PEG-40 hydrogenated | 0.5 | 0.5 |
| castor oil | | |
| Glycerin | 0.1 | 0.1 |
| Ethanol | 15 | 15 |
| Water | Qs 100 | Qs 100 |

(1) Ecoskin RS (Solabia)
(2) Firmalift GRV (Solabia): Extract of yeast Saccharomyces cerevisiae in a water/pentylene glycol mixture
(3) Repair Complex CLR PF (CLR)

The composition C according to the invention is visually clear while the comparative composition C′ is opaque.

In addition, composition C is more fluid than composition C′, therefore composition C will be easier to distribute on the hair and scalp than composition C′.

Natural locks of hair (5.4 g) were previously washed with a DOP chamomile shampoo at a rate of 0.4 g of shampoo par g of hair, followed by a rinse.

Each of the compositions E and E′ were applied to the wet locks of natural hair at a rate of 0.15 g of composition per g of hair. Then the locks were dried with a hair dryer (without being rinsed).

Then, 6 experts evaluated visually, and comparatively, the volume conferred to the hair on a scale ranging from 0 (no volume, hair is tightly packed together and the lock takes up little volume in the space) to 5 (a lot of volume, hair is loose from each other, the lock takes up a lot of volume in the space)

The following results were obtained:

TABLE 6

| | Composition C | Composition C′ |
|---|---|---|
| | (invention) | (comparative) |
| Expert 1 | 3 | 2 |
| Expert 2 | 3 | 2 |
| Expert 3 | 3 | 2 |
| Expert 4 | 4 | 2 |
| Expert 5 | 4 | 2 |
| Expert 6 | 4 | 3 |
| Mean value | 3.5 | 2.1 |
| standard | 0.5 | 0.4 |
| deviation | | |

Composition C according to the invention gave significantly more volume to the hair compared to the comparative composition C′.

According to all the experts, the locks treated with the composition C according to the invention have a more natural feel and a cleaner look than the locks treated with comparative composition C′.

1. Composition comprising:
(i) one or more antioxidants chosen from ascorbic acid and its derivatives,
(ii) one or more oxyalkylenated esters of fatty acids and of sorbitan,
(iii) one or more microorganisms of the genus Bifidobacterium species, one of its fractions, one of its metabolites or their mixtures, the microorganism(s) being in the lysate form, and
(iv) one or more extracts of yeast of the genus Saccharomyces.

2. Composition according to claim 1, characterized in that the antioxidant (i) is ascorbyl glucoside.

3. Composition according to claim 1, characterized in that the total content of the antioxidant(s) (i) ranges from 0.5% to 10% by weight, and preferably from 0.7% to 5% by weight, more preferentially from 0.8% to 3% by weight, with respect to the total weight of the composition.

4. Composition according to claim 1, characterized in that the oxyalkylenated ester(s) of fatty acids and of sorbitan (ii) are chosen from esters of saturated or unsaturated, linear or branched, C₈ to C₃₀ fatty acids and of sorbitan, comprising from 1 to 20 oxyethylene units, and their mixtures; preferably from esters of C₈ to Cis fatty acid and of sorbitan, comprising from 4 to 20 oxyethylene units, and their mixtures, more preferentially from esters of preferably linear and saturated C₁₀-C₁₄ fatty acid and of sorbitan, comprising from 4 to 10 oxyethylene units; more preferentially still from polyoxyethylene (4) sorbitan monolaurate, polyoxyethylene (20) sorbitan monolaurate, polyoxyethylene (20) sorbitan monopalmitate, polyoxyethylene (20) sorbitan monostearate, polyoxyethylene (4) sorbitan monostearate, polyoxyethylene (20) sorbitan tristearate, polyoxyethylene (20) sorbitan monooleate, polyoxyethylene (5) sorbitan monooleate and polyoxyethylene (20) sorbitan trioleate; better still from polyoxyethylene (4) sorbitan monolaurate, polyoxyethylene (20) sorbitan monolaurate and their mixtures; even better still, the oxyalkylenated ester of fatty acid and of sorbitan (ii) is polyoxyethylene (4) sorbitan monolaurate.

5. Composition according to claim 1, characterized in that the total content of the oxyalkylenated ester(s) of fatty acids and of sorbitan (ii) ranges from 0.05% to 10% by weight, preferably from 0.1% to 5% by weight, more preferentially from 0.2% to 2% by weight, with respect to the total weight of the composition.

6. Composition according to claim 1, characterized in that the microorganism(s) (iii) of the genus Bifidobacterium species are chosen from the following species: Bifidobacterium longum, Bifidobacterium bifidum, Bifidobacterium breve, Bifidobacterium animalis, Bifidobacterium lactis, Bifidobacterium infantis, Bifidobacterium adolescentis, Bifidobacterium pseudobacterium and their mixtures; preferably, the microorganism (iii) is of the species Bifidobacterium longum.

7. Composition according to claim 1, characterized in that the total content, expressed as dry extract, of the lysate of the microorganism(s) (iii) ranges from 0.001% to 20% by weight, preferably from 0.01% to 10% by weight, more preferentially from 0.05% to 5% by weight, better still from 0.1% to 2% by weight, with respect to the total weight of the composition.

8. Composition according to claim 1, characterized in that the yeast(s) (iv) of the genus Saccharomyces are chosen from the following species: Saccharomyces bailii, Saccharomyces carlsbergensis, Saccharomyces uvarum, Saccharomyces cerevisiae, Saccharomyces delbrueckii, Saccharomyces exiguus, Saccharomyces fermentati, Saccharomyces florentinus, Saccharomyces fragilis, Saccharomyces fructuum, Saccharomyces heterogenicus, Saccharomyces oleaginosus, Saccharomyces rosei, Saccharomyces steineri, Saccharomyces boulardii, Saccharomyces kefir, Saccharomyces kluyveri and their mixtures; and preferably the yeast of the genus Saccharomyces is a yeast of the species Saccharomyces cerevisiae.

9. Composition according to claim 1, characterized in that the total content (as dry matter or active material) of the extract(s) of yeast (iv) of the genus Saccharomyces ranges from 0.001% to 5% by weight, preferably from 0.01% to 1% by weight, more preferentially from 0.02% to 0.5% by weight, with respect to the total weight of the composition.

10. Composition according to claim 1, characterized in that it additionally comprises one or more monosaccharide(s), preferably chosen from mannose, rhamnose, and also their α or β anomers, their optical isomers of L or D configuration, their solvates and their mixtures, more preferentially from mannose, and also its α or β anomers, its optical isomers of L or D configuration, its solvates and their mixtures; better still, the monosaccharide is D-mannose.

11. Composition according to claim 10, characterized in that the total content of the monosaccharide(s) ranges from 0.01% to 20% by weight, preferably from 0.05% to 10% by weight, and more preferentially from 0.1% to 5% by weight, with respect to the total weight of the composition.

12. Composition according to claim 1, characterized in that it also comprises one or more additional saccharides, different from the monosaccharides, chosen from oligosaccharides, polysaccharides and their mixtures, preferably chosen from inulin, fructooligosaccharide, glucooligosaccharide, oligosaccharides derived from soybeans, pyrodextrins, isomaltooligosaccharide, xylooligosaccharide, transgalactooligosaccharide and their mixtures.

13. Composition according to claim 12, characterized in that the additional saccharide(s) correspond to a mixture comprising at least one fructooligosaccharide and at least one glucooligosaccharide.

14. Composition according to claim 12, characterized in that the total content of the additional saccharide(s) ranges from 0.01% to 20% by weight, preferably from 0.05% to 20% by weight, more preferentially from 0.05% to 10% by weight, better still from 0.05% to 5% by weight, even better still from 0.1% to 2% by weight, with respect to the total weight of the composition.

15. Composition according to claim 1, characterized in that it additionally comprises one or more probiotic microorganisms, different from the microorganisms of the genus Bifidobacterium species, preferably chosen from probiotic microorganisms from the group of the lactic acid bacteria and their mixtures, and more preferentially from Lactobacillus acidophilus, Lactobacillus alimentarius, Lactobacillus curvatus, Lactobacillus delbruckii subsp. lactis, Lactobacillus gasseri, Lactobacillus johnsonii, Lactobacillus reuteri, Lactobacillus casei, Lactobacillus rhamnosus (Lactobacillus GG), Lactobacillus sake, Lactobacillus lactis, Streptococcus thermophilus, Lactobacillus delbrueckii, Lactobacillus helveticus, Lactobacillus salivarius, Lactobacillus plantarum, Lactobacillus sakei, Lactobacillus brevis, Lactobacillus buchneri, Lactobacillus fermentum, Lactobacillus bulgaricus, Lactobacillus longum and their mixtures.

16. Composition according to claim 15, characterized in that the total content of the probiotic microorganism(s), different from the microorganisms of the genus Bifidobacterium species, ranges from 0.0001% to 20% by weight, preferably from 0.0001% to 10% by weight, more preferentially from 0.001% to 5% by weight, better still from 0.001% to 1% by weight, with respect to the total weight of the composition.

17. Composition according to claim 1, characterized in that it additionally comprises one or more non-ionic surfactants, different from the oxyalkylenated ester(s) of C₈ to C₃₀ acids and of sorbitol (ii), preferably chosen from saturated or unsaturated, linear or branched, oxyalkylenated or glycerolated, preferentially oxyethylenated, C₈ to C₄₀ alcohols; saturated or unsaturated oxyethylenated vegetable oils comprising from 1 to 100 mol of ethylene oxide, preferably from 2 to 50, and their mixtures.

18. Method for the cosmetic treatment of keratin materials, in particular human keratin materials, such as the scalp and the hair, comprising the application, to said keratin materials, of a composition as defined in claim 1.

19. Non-therapeutic cosmetic use of a composition as defined in claim 1 for maintaining, indeed even restoring, the microbial flora of the skin and of the scalp.

20. Device comprising one or more containers which comprises (comprise) a composition as defined in claim 1.
